(19) United States
(12) Patent Application Publication
(10) Pub. No.: US 2025/0179463 A1
(43) Pub. Date: Jun. 5, 2025

(54) HDAC4-MEDIATED NEUROPROTECTION

(71) Applicants: The Board of Trustees of the Leland Stanford Junior University, Stanford, CA (US); The United States Government as Represented By The Department of Veterans Affairs, NW Washington, DC (US)

(72) Inventors: Michael Seth Kapiloff, Los Altos, CA (US); Jeffrey Louis Goldberg, Menlo Park, CA (US); Ying Zhu, Redwood City, CA (US)

(21) Appl. No.: 18/876,632

(22) PCT Filed: Jun. 26, 2023
(86) PCT No.: PCT/US2023/026224

§ 371 Date: Dec. 18, 2024

Related U.S. Application Data
(60) Provisional application No. 63/358,429, filed on Jul. 5, 2022.

(51) Int. Cl. C12N 9/80; A61K 9/00; A61K 48/00; A61P 27/06; C12N 15/86

(52) U.S. Cl. CPC C12N 9/80; A61K 9/0048; A61K 48/0041; A61P 27/06; C12N 15/86; C12N 2750/14143; C12Y 305/01098

(57) ABSTRACT

Methods and compositions are provided for the treatment of glaucoma and other optic neuropathies by administering an effective dose of an HDAC4 coding sequence or protein.

### STATEMENT REGARDING FEDERALLY SPONSORED RESEARCH

This invention was made with Government support under contract EY031167 awarded by the National Institutes of Health. The Government has certain rights in the invention.

### BACKGROUND

The forms of glaucoma are a group of optic neuropathies characterized by progressive degeneration of retinal ganglion cells. These are central nervous system neurons that have their cell bodies in the inner retina and axons in the optic nerve. Degeneration in glaucoma leads to visual loss. The biological basis of glaucoma is poorly understood and the factors contributing to its progression have not been fully characterized.

Glaucoma affects more than 70 million people worldwide with approximately 10% being bilaterally blind, see Quigley et al. (2006) Br J Ophthalmol. 90(3):262-267, making it the leading cause of irreversible blindness in the world. Glaucoma can remain asymptomatic until it is severe, resulting in a high likelihood that the number of affected individuals is much higher than the number known to have it. Population-level surveys suggest that only 10% to 50% of people with glaucoma are aware they have it. Forms of glaucoma can be classified into 2 broad categories: open-angle glaucoma and angle-closure glaucoma. In the United States, more than 80% of cases are open-angle glaucoma; however, angle-closure glaucoma is responsible for a disproportionate number of patients with severe vision loss. Both open-angle and angle-closure glaucoma can be primary diseases. Secondary glaucoma can result from trauma, certain medications such as corticosteroids, inflammation, tumor, or conditions such as pigment dispersion or pseudo-exfoliation.

Therapeutic methods to alleviate glaucoma are of great clinical interest and addressed by this. The methods to alleviate glaucoma may also be useful for a variety of clinical conditions, including traumatic optic nerve injury, optic neuritis, ischemic nerve injury, Alzheimer's disease, stroke, and other conditions in which death of neurons contributes to the disorder.

### SUMMARY

Methods and compositions are provided for the treatment of glaucoma and other optic neuropathies. In such methods, an effective dose of a composition comprising a polynucleotide sequence encoding nuclear-localized histone deacetylase II (HDAC4) polypeptide or an N-terminal fragment thereof, is administered to the individual for prevention or treatment of glaucoma or other optic neuropathies. Provided herein are data demonstrating that such HDAC4 proteins or a fragment thereof have an effect on RGC regeneration after injury.

In some embodiments the HDAC4 polypeptide is a nuclear-localized polypeptide comprising amino acid substitutions at one or more residues selected from S246, S467 and S632, where the amino acid substitution is to an amino acid other than serine. In some embodiments the substitution is to an alanine, i.e. S246A, S467A, S632A. In some embodiments a nuclear-localized, phosphoablative HDAC4 mutant comprises each of the amino acid substitutions S246A, S467A, S632A, which mutant may be referred to as “3SA”.

In some embodiments the HDAC4 polypeptide is an N terminal fragment of HDAC4. HDAC4 is proteolytically processed by a PKA dependent mechanism to yield an active N-terminal fragment (HDAC4-NT). HDAC4-NT is a constitutive repressor of MEF2-dependent gene expression. There is increased RGC survival following HDAC4-NT expression following injury. In some embodiments the HDAC4-NT comprises an HDAC4 protein truncated at about residue 201.

In some embodiments the polynucleotide encoding HDAC4 is operably linked to a promoter active in retinal cells, e.g. retinal ganglion cells. In some embodiments the HDAC4 polynucleotide sequence is a human HDAC4 sequence. In some embodiments, delivery of the sequence is intravitreal. The polynucleotide sequence may be provided in a suitable viral vector, e.g. an AAV vector.

In some embodiments, the disclosure provides genetic sequences encoding human HDAC4 proteins or a fragment thereof as described above for promoting axon regeneration of retinal ganglion cells (RGCs) that have been damaged, or are susceptible to damage, as a result of glaucoma or other optic neuropathies, to an individual in need thereof. In some embodiments, the RGCs are human RGCs. In some embodiments the HDAC coding sequences are provided in a viral vector, e.g. an AAV vector. In some embodiments the sequences are provided in a composition formulated for injection. In some embodiments, the composition is formulated for intraocular injection, subretinal injection, intravitreal injection, periocular injection, subconjunctival injection, retrobulbar injection, intracameral injection, or sub-Tenon's injection. Alternatively the formulation is administered parenterally.

In some embodiments, a method is provided for treating or preventing glaucoma or other optic neuropathies in an individual in need thereof, the method comprising administering to the individual a therapeutically effective amount of an injectable composition, comprising, consisting or consisting essentially of: (a) one or both of a polynucleotide sequence encoding human HDAC4 proteins or a fragment thereof; and (b) a pharmaceutically acceptable diluent, excipient, vehicle, or carrier. In some embodiments the injectable composition consists essentially of (a) a sequence encoding HDAC4 proteins or a fragment thereof; (b) an additional therapeutic agent; and (c) a pharmaceutically acceptable diluent, excipient, vehicle, or carrier. The HDAC4 sequence may be HDAC4 3SA. The HDAC4 sequence may be HDAC4-NT. The HDAC4 sequence may be operably joined to a promoter. The HDAC4 sequence may be provided in a viral, e.g. an AAV vector, such as AAV2. In some embodiments, the additional therapeutic agent is an additional agent for treating glaucoma and other optic neuropathies. In some embodiments, the additional therapeutic agent is selected from: aunorubicin, retinoic acid, 5-fluorouracil, intravitreal triamcinolone acetonide, ranibizumab, bevacizumab, dasatinib, pegaptanib sodium, N-acetyl-cysteine (NAC), pioglitazone, glucosamine, genistin, geldanamycin, fausdil, resveratrol, pentoxyfilline, dipyridamole, a corticosteroid, and/or an antioxidant. In some embodiments, the therapeutically effective amount is effective for promoting the growth of RGC axons.

### BRIEF DESCRIPTION OF DRAWINGS

FIG. 1. Model for HDAC4-mediated RGC neuroprotection. HDAC4 translocates into the nucleus, promoting histone deacetylation and in some cases MEF2a sumoylation, thereby repressing MEF2a regulated genes and protecting RGCs after injury.

FIG. 2. Requirement of HDACs for RGC survival after optic nerve crush injury. AAV2 small hairpin RNA (shRNA) viruses specific for individual HDAC family members were intravitreally injects 2 weeks prior to optic nerve crush in S129 mice. RGC survival was assayed by flat-mount RBPMS immunohistochemistry (**, p<0.01).

FIGS. 3A-3D. An HDAC4 3SA mutant is constitutively nuclear in RGCs. (A) AAV2 was used to expressed Flag-tagged wildtype and phosphoablative 3SA HDAC4 mutant. Retinal flat mounts were stained 2 weeks after AAV2 intravitreal injection. RBPMS and Tuj1 are markers for retinal ganglion cells. Hoechst stains the nucleus. (B) HDAC4 domain structure. MEF2 binding site and PKD phosphorylation sites S246/467/632 are indicated. NLS and NES-nuclear localization and export signals. (C) AAV2.hHDAC4.3SA that expressed the constitutively nuclear Flag-HDAC4 3SA mutant. (D) Flag-tagged HDAC4 localization was determined by relative immunofluorescence for the cytosol and nucleus using Image J. following background subtraction. (Scale bar: 50 μm. **, p<0.01).

FIGS. 4A-4D. HDAC4 3SA protects RGCs and promotes axon regeneration after optic nerve crush injury. (A) Representative images showing RGC survival after crush or sham surgery In C57BL6/J mice intravitreally injected with AAV2.HDAC4 WT, AAV2.HDAC4 3SA, or AAV2.GFP control. (B) Quantification of RGC survival 2 weeks after optic nerve crush. **, p<0.01. (C) Cholera toxin staining of optic nerves. (D) Axon regeneration distal to the crush site. (scale bar: A, 50 μm, C, 100 μm. *, p<0.05).

FIGS. 5A-5D. HDAC4 NT protects RGCs and promotes axon regeneration after optic nerve crush injury. (A) Representative images showing RGC survival after crush or sham surgery in C57BL6/J mice intravitreally injected with AAV2.HDAC4-NT, AAV2.GFP control or no AAV. (B) Quantification of RGC survival 2 weeks after optic nerve crush. (C) Cholera toxin staining of optic nerves. (D) Axon regeneration distal to the crush site. (scale bar: A, 50 μm, C, 100 μm. *, p<0.05).

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any way.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, B-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, SiRNA, shRNA, guide RNA, anti-sense RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

By the terms “effective amount” and “therapeutically effective amount” of a formulation or formulation component is meant a sufficient amount of the formulation or component, alone or in a combination, to provide the desired effect. For example, by “an effective amount” is meant an amount of a human HDAC4 protein or a fragment thereof, sufficient to treat or prevent glaucoma or other optic neuropathies in a mammal. Ultimately, the attending physician or veterinarian decides the appropriate amount and dosage regimen.

The effective dose of a therapeutic composition to be given to a particular patient will depend on a variety of factors, several of which will be different from patient to patient. Utilizing ordinary skill, the competent clinician will be able to optimize the dosage of a particular therapeutic or imaging composition in the course of routine clinical trials. The agent is administered at a dosage, alone or in combination with other agents, that enhances neuron recovery while minimizing any side-effects. The effectiveness of recovery may be assessed, for example, by monitoring function of the neuron, e.g. maintenance or recovery of vision in glaucoma or other optic neuropathy patients, such as at least about 5% recovery, at least about 10% recovery, at least about 20%, at least about 30%, at least about 40%, at least about 50%, at least about 75%, at least about 85%, at least about 95% or more, e.g. assessing by conventional measures of vision or retinal structure. It is contemplated that compositions will be obtained and used under the guidance of a physician for in vivo use. The dosage of the therapeutic formulation will vary widely, depending upon the nature of the disease, the frequency of administration, the manner of administration, the clearance of the agent from the host, and the like.

In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% decrease in loss of function, e.g. visual acuity, relative to function measured in absence of administering the human HDAC4 nucleic acid or polypeptide or a fragment thereof. Treatment may result in at least about a 10%, 15%, 20%, 25%, 30%, 35%, 40%, 45%, 50%, 55%, 60%, 65%, 70%, 75%, 80%, 85%, 90%, 95%, or even 100% decrease in symptoms of glaucoma or other optic neuropathies, compared to a subject that is not treated with a human HDAC4 nucleic acid or polypeptide or a fragment thereof.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical formulation that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical formulation” is meant to encompass a formulation suitable for administration to a subject, such as a mammal, especially a human. In general, a “pharmaceutical formulation” is sterile, and preferably free of contaminants that are capable of eliciting an undesirable response within the subject (e.g., the compound(s) in the pharmaceutical formulation is pharmaceutical grade). Pharmaceutical formulations can be designed for administration to subjects or patients in need thereof via a number of different routes of administration including intravitreal, subretinal, oral, buccal, rectal, parenteral, intraperitoneal, intradermal, intratracheal, intramuscular, subcutaneous, and the like.

As used herein, the term “administration” refers to the administration of a formulation or composition (i.e. a composition comprising a polynucleotide encoding human HDAC4 or a fragment thereof) to a subject or system. Administration to an animal subject (e.g., to a human) may be by any appropriate route. For example, in some embodiments, administration may be bronchial (including by bronchial instillation), buccal, enteral, intradermal, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (including by intratracheal instillation) transdermal, vaginal, intravitreal and subretinal. In some embodiments, administration may involve intermittent dosing. In some embodiments, administration may involve continuous dosing (e.g., perfusion) for at least a selected period of time.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bolag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Glaucoma: An eye disorder characterized by retinal ganglion cell death, excavation of the optic nerve head and gradual loss of the visual field. An abnormally high intraocular pressure is commonly known to be detrimental to the eye and is one of the main risk factors in glaucoma. In glaucoma patients, high intraocular pressure can result in degenerative changes in the retina. “Ocular hypertension” refers to clinical situation in individuals with an abnormally high intraocular pressure without any manifestation of defects in the visual field or optic nerve head. Individuals with ocular hypertension carry the risk of conversion to glaucoma with the risk being correlated to higher intraocular pressure measurements.

Glaucoma can be divided into open-angle form and the closed-angle forms and further classified into acute and chronic forms. There also is a normal-tension glaucoma. The glaucoma can be a primary or a secondary glaucoma. More than 80% of all glaucoma cases are chronic open angle glaucoma (COAG), also called primary open angle glaucoma. Any of these forms of glaucoma can be treated using the methods disclosed herein.

“Primary angle closure glaucoma” is caused by contact between the iris, trabecular meshwork, and peripheral cornea which in turn obstructs outflow of the aqueous humor from the eye. This contact between iris and trabecular meshwork (TM) may gradually damage the function of the meshwork until it fails to keep pace with aqueous production, and the pressure rises. In over half of all cases, prolonged contact between iris and TM causes the formation of synechiae (effectively “scars”). These cause permanent obstruction of aqueous outflow. In some cases, pressure may rapidly build up in the eye, causing pain and redness (symptomatic, or so-called “acute” angle closure). In this situation, the vision may become blurred, and halos may be seen around bright lights. Accompanying symptoms may include a headache and vomiting. Diagnosis can made from physical signs and symptoms: pupils mid-dilated and unresponsive to light, cornea edematous (cloudy), reduced vision, redness, and pain. However, the majority of cases are asymptomatic. Prior to the very severe loss of vision, these cases can only be identified by examination, generally by an eye care professional.

“Primary open-angle glaucoma” occurs when optic nerve damage results in a progressive loss of the visual field. Not all people with primary open-angle glaucoma have eye pressure that is elevated beyond normal. The increased pressure is caused by the blockage of the aqueous humor outflow pathway. Because the microscopic passageways are blocked, the pressure builds up in the eye and causes imperceptible very gradual vision loss. Peripheral vision is affected first, but eventually the entire vision will be lost if not treated. Diagnosis can be made by looking for cupping of the optic nerve and measuring visual field. Prostaglandin agonists work by opening uveoscleral passageways.

Other forms of glaucoma are developmental glaucoma and secondary glaucoma, which can occur after uveitis, iridocyclitis, intraocular hemorrhage, trauma, or an intraocular tumor. Any form of glaucoma can be treated using the methods disclosed herein.

The death of retinal ganglion cells occurs in glaucoma. Methods are disclosed herein for increasing the survival of retinal ganglion cells.

By “neurological” or “cognitive” function as used herein, it is meant the patient's ability to think, function, etc. In conditions where there is axon loss and regrowth, there may be recovery of motor and/or sensory abilities.

By “neurodegenerative disease, disorder, or condition” is meant a disease, disorder, or condition (including a neuropathy) associated with degeneration or dysfunction of neurons or other neural cells throughout the nervous system, including but not limited to those in the retina such as retinal ganglion cells or photoreceptor cells. A neurodegenerative disease, disorder, or condition can be any disease, disorder, or condition in which decreased function or dysfunction of neurons, or loss or neurons or other neural cells, can occur.

As used herein, a “neuron or portion thereof” can consist of or be a portion of a neuron, for example a retinal ganglion cell, and the like. More particularly, the term “neuron” as used herein denotes nervous system cells that include a central cell body or soma, and two types of extensions or projections: dendrites, by which, in general, the majority of neuronal signals are conveyed to the cell body; and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons).

In some embodiments, the neuron or portion thereof can be present in a subject, such as a human subject. The subject can, for example, have or be at risk of developing a disease, disorder, or condition of the nervous system, an injury to the nervous system, such as, for example, an injury caused by physical, mechanical, or chemical trauma; ocular-related neurodegeneration; and the like. By “neurodegenerative disease, disorder, or condition” is meant a disease, disorder, or condition (including a neuropathy) associated with degeneration or dysfunction of neurons or other neural cells, such as retinal ganglion cells or photoreceptor cells. Examples of ocular-related neurodegeneration include, but are not limited to, glaucoma, retinitis pigmentosa, age-related macular degeneration (AMD), photoreceptor degeneration associated with wet or dry AMD, other retinal degeneration, optic nerve drusen, ischemic or traumatic optic neuropathy, and optic neuritis.

Examples of injuries to the nervous system caused by physical, mechanical, or chemical trauma include, but are not limited to, nerve damage caused by ischemia, exposure to toxic compounds, heavy metals (e.g., lead, arsenic, and mercury), industrial solvents, drugs, chemotherapeutic agents, dapsone, HIV medications (e.g., zidovudine, didanosine, stavudine, zalcitabine, ritonavir, and amprenavir), cholesterol lowering drugs (e.g., lovastatin, indapamide, and gemfibrozil), heart or blood pressure medications (e.g., amiodarone, hydralazine, perhexiline), and metronidazole. More particularly, traumatic injury or other damage to neuronal cells (e.g., trauma due to accident, blunt-force injury, gunshot injury, spinal cord injury, ischemic conditions of the nervous system such as stroke, cell damage due to aging or oxidative stress, and the like) also is intended to be included within the language “neurodegenerative disease, disorder, or condition.” In such embodiments, the presently disclosed methods can be used to treat neuronal damage due to traumatic injury or stroke by preventing death of damaged neuronal cells and/or by promoting or stimulating neurite growth from damaged neuronal cells.

Further examples also include burn, wound, surgery, accidents, ischemia, prolonged exposure to cold temperature, stroke, intracranial hemorrhage, and cerebral hemorrhage. More particularly, traumatic injury or other damage to neuronal cells, e.g., trauma due to accident, blunt-force injury, gunshot injury, spinal cord injury, ischemic conditions of the nervous system such as stroke, cell damage due to aging or oxidative stress, and the like is also included within the language “neurodegenerative disease, disorder, or condition.” In such embodiments, the presently disclosed methods can be used to treat neuronal damage due to traumatic injury or stroke by preventing death of damaged neuronal cells and/or by promoting or stimulating neurite growth from damaged neuronal cells.

In some embodiments, the subject is suffering from or susceptible to a neurodegenerative disease, disorder, or condition, such as glaucoma, e.g., a subject diagnosed as suffering from or susceptible to a neurodegenerative disease, disorder, or condition. In other embodiments, the subject has been identified (e.g., diagnosed) as suffering from or susceptible to a neurodegenerative disease, disorder, or condition (including traumatic injury) in which neuronal cell loss is implicated, or in which damage to neurites is involved, and for which treatment or prophylaxis is desired.

In some embodiments, the presently disclosed methods include preventing or inhibiting neuron or axon degeneration. Preventing axon or neuron degeneration includes decreasing or inhibiting axon or neuron degeneration, which may be characterized by complete or partial inhibition of neuron or axon degeneration. Such prevention or inhibition can be assessed, for example, by analysis of neurological function. Further, the phrases “preventing neuron degeneration” and “inhibiting neuron degeneration” include such inhibition with respect to the entire neuron or a portion thereof, such as the neuron cell body, axons, and dendrites.

HDAC4. Histone deacetylase 4, also known as HDAC4 belongs to class II of the histone deacetylase/AcuC/AphA family. It possesses histone deacetylase activity and represses transcription when tethered to a promoter. This protein does not bind DNA directly but through transcription factors such as MEF2, SRF, Runx-2, and CREB. It can function as part of a multiprotein complex with RbAp48, NCoR, SMRT, and HDAC3. The refseq for human HDAC4 can be accessed at Genbank, NM_006037; NM_001378414; NM_001378415; NM_001378416; NM_001378417; NP_006028; NP_001365343; NP_001365344; NP_001365345; NP_001365346.

An exemplary reference sequence of wild-type HDAC4 has the sequence (SEQ ID NO:1), which may be used as a reference for amino acid modifications and truncation:

| | |
|---|---|
| 1 | mssqshpdgl sgrdqpvell nparvnhmps tvdvatalpl qvapsavpmd lrldhqfslp |
| 61 | vaepalreqq lqqellalka kqqiqrqili aefqrqheql srqheaqlhe hikqqqemla |
| 121 | mkhqqelleh qrklerhrqe qelekqhreq klqqlknkek gkesavaste vkmklqefvl |
| 181 | nkkkalahrn lnhcissdpr ywygktqhss ldqssppqsg vstsynhpvl gmydakddfp |
| 241 | lrktasepnl klrsrlkqkv aerrsspllr rkdgpvvtal kkrpldvtds acssapgsgp |
| 301 | sspnnssgsv saengiapav psipaetsla hrlvaregsa aplplytsps lpnitlglpa |
| 361 | tgpsagtagq qdaerltlpa lqqrlslfpg thltpylsts plerdggaah spllqhmvll |
| 421 | eqppaqaplv tglgalplha qslvgadrvs psihklrqhr plgrtqsapl pqnaqalqhl |
| 481 | viqqqhqqfl ekhkqqfqqq qlqmnkiipk pseparqpes hpeeteeelr ehqalldepy |
| 541 | ldrlpgqkea haqagvqvkq epiesdeeea epprevepgq rqpseqellf rqqallleqq |
| 601 | rihqlrnyqa smeaagipvs fgghrplsra qsspasatfp vsvqepptkp rfttglvydt |
| 661 | lmlkhqctcg sssshpehag riqsiwsrlq etglrgkcec irgrkatlee lqtvhseaht |
| 721 | llygtnplnr qkldskkllg slasvfvrlp cggvgvdsdt iwnevhsaga arlavgcvve |
| 781 | lvfkvatgel kngfavvrpp ghhaeestpm gfcyfnsvav aakllqqrls vskilivdwd |
| 841 | vhhgngtqqa fysdpsvlym slhryddgnf fpgsgapdev gtgpgvgfnv nmaftggldp |
| 901 | pmgdaeylaa frtvvmpias efapdvvlvs sgfdaveghp tplggynlsa rcfgyltkql |
| 961 | mglaggrivl alegghdlta icdaseacvs allgneldpl pekvlqqrpn anavrsmekv |
| 1021 | meihskywrc lqrttstagr slieaqtcen eeaetvtama slsvgvkpae krpdeepmee |
| 1081 | eppl |

In some embodiments the HDAC4 protein comprises amino acid substitutions at one or more residues selected from S246, S467 and S632, where the amino acid substitutions are to an amino acid other than serine. In some embodiments the substitution is to an alanine, i.e. S246A, S467A, S632A. In some embodiments a nuclear localized, phosphoablative HDAC4 mutant comprises each of the amino acid substitutions S246A, S467A, S632A, which mutant may be referred to as “3SA”.

In some embodiments an HDAC4 N terminal fragment is used. HDAC4 can be proteolytically processed by a PKA dependent mechanism to yield an active N-terminal fragment (HDAC4-NT) that includes HDAC4 residues aa 1-201. In some embodiments the HDAC4-NT comprises an HDAC4 protein truncated at about residue 201. HDAC4-NT is a constitutive repressor of MEF2-dependent gene expression. There is greater RGC survival following HDAC4-NT expression following an injury.

The sequence of an HDAC4 protein may be altered in various ways known in the art to generate targeted changes in sequence. The polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

### Promoters and Vectors

Aspects of the present invention disclose expression cassettes and/or vectors comprising polynucleotides. Suitably, the polynucleotides can comprise promoters operably linked to the region of the polynucleotide that encodes a human HDAC4 protein or fragment thereof. Virtually any promoter capable of driving these polynucleotides can be used.

Targeted expression can be accomplished using a general promoter, or a cell specific promoter. Examples of cell specific promoters are promoters for somatostatin, parvalbumin, GABAa6, L7, and calbindin. Other cell specific promoters can be promoters for kinases such as PKC, PKA, and CaMKII; promoters for other ligand receptors such as NMDAR1, NNIDAR2B, GluR2; promoters for ion channels including calcium channels, potassium channels, chloride channels, and sodium channels; and promoters for other markers that label classical mature and dividing cell types, such as calretinin, nestin, and beta3-tubulin.

Specifically, where expression of a subject polynucleotide in a retinal ganglion cell is desired, a promoter of interest may be used. Promoters of interest include but are not limited to, NEFH promoter, gamma-synuclein promoter, synapsin-1 promoter, a THY1 promoter, etc., whether of human origin or other species.

Variants of the above discussed promoters may also be used. In some instances, a suitable variant comprises a nucleotide sequence having 60% or more, 70% or more, 75% or more, 80% or more, 85% or more, 90% or more, 95% or more, 99% or more or 100% nucleotide sequence identity to their corresponding “reference”, or wild-type, promoter. A person of skill in the art will recognize that various promoters drive expression in various cell types, and will be able to decide on which promoter to use for their desired outcome.

In some embodiments, the vector is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV can infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87:2211-2215; Samulski et al, EMBO J., 1991. 10:3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134: 52-63), while AAV1-4 and AAV6 are all found in the study of adenovirus (Ursula Bantel-Schaal, Hajo Delius and Harald zur Hausen. J. Viral., 1999. 73: 939-947).

AAV vectors may be prepared using any convenient methods. Adeno-associated viruses of any serotype are suitable, although AAV2 may be preferred (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. ME Bloom, RM Linden, C RParrish, Eds.) p 5-14, Rudder Arnold, London, UK (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118; 6,989,264; and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs used in the methods of the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535. AAV2 may be preferred.

In some embodiments, the HDAC4 sequence within the therapeutic nucleic acid sequence encodes one or more elements of a programmable gene editing system, e.g. a CRISPR/Cas9 system. In other embodiments, the HDAC4 sequence may comprise, but not be limited to, recombinant DNA, shRNA, miRNA, etc.

In some embodiments, the AAV vector comprises AAV2 inverted terminal repeats (ITRs). In some embodiments, other AAV ITRs may be used which include but are not limited to AAV1, AAV2, AAV3, AAV4, AAV6, AAV7, AAV8, AAV9, etc.

In an embodiment, the nucleic acid composition is packaged in ultra-purified viral particle. In some embodiments, the viral capsid (or particle) is an AAV5 or an AAV2 viral capsid. Ultra-purified refers to the level of contamination present within a composition. In some embodiments, ultra-purified means that the level of contamination is at least <0.01% of the total solution. In some embodiments, ultra-purified refers to a level of contamination that is 0.01-0.005%, 0.005-0.001%, 0.001-0.0005%, 0.0005-0.0001%, or <0.0001% of the total composition. In some embodiments, other AAV viral capsids may be used which include but are not limited to AAV1, AAV2, AAV3, AAV4, AAV6, AAV7, AAV8, AAV9, etc.

In some embodiments, an effective dose for efficient genetic modification comprises at least >10⁸ viral particles/ml. In some embodiments, an effective dose comprises 10⁸-10⁹ viral particles/mL, 10⁹-10¹⁰ viral particles/ml, 10¹⁰-10¹¹ viral particles/ml, 10¹¹-10¹² viral particles/ml, 10¹²-10¹³ viral particles/ml or >10¹³ viral particles/ml. An effective dose may be from about 0.5 to 10 μl/injection/eye, e.g. from about 1 to 10, from about 1-5, from about 2-8 μl/injection/eye.

In an embodiment, the HDAC4 sequence is packaged in a single stranded, double stranded, or self-complementary AAV vector.

In some embodiments, a human HDAC4 nucleic acid or polypeptide or a fragment thereof binds to a class 2 CRISPR/Cas effector protein (e.g., a Cas9 protein; a type V or type VI CRISPR/Cas protein; a Cpf1 protein; etc.) and targets the complex to a specific location within a target nucleic acid, which is referred to herein as a “guide RNA” or “CRISPR/Cas guide nucleic acid” or “CRISPR/Cas guide RNA.” For the purpose of modifying the native HDAC4 locus, e.g. to truncate the HDAC4 polypeptide, or to provide for amino acid substitutions. A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

A guide RNA can be referred to by the protein to which it corresponds. For example, when the class 2 CRISPR/Cas effector protein is a Cas9 protein, the corresponding guide RNA can be referred to as a “Cas9 guide RNA.” Likewise, as another example, when the class 2 CRISPR/Cas effector protein is a Cpf1 protein, the corresponding guide RNA can be referred to as a “Cpf1 guide RNA.”

In some embodiments, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some embodiments, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, a nucleic acid payload includes or encodes a gene editing tool (i.e., a component of a gene editing system, e.g., a site specific gene editing system such as a programmable gene editing system). For example, a nucleic acid payload can include one or more of: (i) a CRISPR/Cas guide RNA, (ii) a DNA encoding a CRISPR/Cas guide RNA, (iii) a DNA and/or RNA encoding a programmable gene editing protein such as a zinc finger protein (ZFP) (e.g., a zinc finger nuclease—ZFN), a transcription activator-like effector (TALE) protein (e.g., fused to a nuclease—TALEN), a DNA-guided polypeptide such as Natronobacterium gregoryi Argonaute (NgAgo), and/or a CRISPR/Cas RNA-guided polypeptide (e.g., Cas9, CasX, CasY, Cpf1, and the like); (iv) a DNA donor template; (v) a nucleic acid molecule (DNA, RNA) encoding a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb1 integrase, R4 integrase, lambda integrase, HK022 integrase, HP1 integrase, and the like); (vi) a DNA encoding a resolvase and/or invertase (e.g., Gin, Hin, yδ3, Tn3, Sin, Beta, and the like); and (vii) a transposon and/or a DNA derived from a transposon (e.g., bacterial transposons such as Tn3, Tn5, Tn7, Tn9, Tn10, Tn903, Tn1681, and the like; eukaryotic transposons such as Tc1/mariner super family transposons, PiggyBac superfamily transposons, hAT superfamily transposons, PiggyBac, Sleeping Beauty, Frog Prince, Minos, Himar1, and the like). In some cases a subject delivery molecule is used to deliver a protein payload, e.g., a gene editing protein such as a ZFP (e.g., ZFN), a TALE (e.g., TALEN), a CRISPR/Cas RNA-guided polypeptide (e.g., Cas9, CasX, CasY, Cpf1, and the like), a site-specific recombinase (e.g., Cre recombinase, Dre recombinase, Flp recombinase, KD recombinase, B2 recombinase, B3 recombinase, R recombinase, Hin recombinase, Tre recombinase, PhiC31 integrase, Bxb1 integrase, R4 integrase, lambda integrase, HK022 integrase, HP1 integrase, and the like), a resolvase/invertase (e.g., Gin, Hin, yδ3, Tn3, Sin, Beta, and the like); and/or a transposase (e.g., a transposase related to transposons such as bacterial transposons such as Tn3, Tn5, Tn7, Tn9, Tn10, Tn903, Tn1681, and the like; or eukaryotic transposons such as Tc1/mariner super family transposons, PiggyBac superfamily transposons, hAT superfamily transposons, PiggyBac, Sleeping Beauty, Frog Prince, Minos, Himar1, and the like). In some cases, the delivery molecule is used to deliver a nucleic acid payload and a protein payload, and in some such cases the payload includes a ribonucleoprotein complex (RNP).

Depending on the nature of the system and the desired outcome, a gene editing system (e.g. a site specific gene editing system such as a programmable gene editing system) can include a single component (e.g., a ZFP, a ZFN, a TALE, a TALEN, a site-specific recombinase, a resolvase/integrase, a transpose, a transposon, and the like) or can include multiple components. In some cases a gene editing system includes at least two components. For example, in some cases a gene editing system (e.g. a programmable gene editing system) includes (i) a donor template nucleic acid; and (ii) a gene editing protein (e.g., a programmable gene editing protein such as a ZFP, a ZFN, a TALE, a TALEN, a CRISPR/Cas RNA-guided polypeptide such as Cas9, CasX, CasY, or Cpf1, and the like), or a nucleic acid molecule encoding the gene editing protein (e.g., DNA or RNA such as a plasmid or mRNA). As another example, in some cases a gene editing system (e.g. a programmable gene editing system) includes (i) a CRISPR/Cas guide RNA, or a DNA encoding the CRISPR/Cas guide RNA; and (ii) a CRISPR/CAS RNA-guided polypeptide (e.g., Cas9, CasX, CasY, Cpf1, and the like), or a nucleic acid molecule encoding the RNA-guided polypeptide (e.g., DNA or RNA such as a plasmid or mRNA). As another example, in some cases a gene editing system (e.g. a programmable gene editing system) includes (i) an NgAgo-like guide DNA; and (ii) a DNA-guided polypeptide (e.g., NgAgo), or a nucleic acid molecule encoding the DNA-guided polypeptide (e.g., DNA or RNA such as a plasmid or mRNA). In some cases a gene editing system (e.g. a programmable gene editing system) includes at least three components: (i) a donor DNA template; (ii) a CRISPR/Cas guide RNA, or a DNA encoding the CRISPR/Cas guide RNA; and (ill) a CRISPR/Cas RNA-guided polypeptide (e.g., Cas9, CasX, CasY, or Cpf1), or a nucleic acid molecule encoding the RNA-guided polypeptide (e.g., DNA or RNA such as a plasmid or mRNA).

As would be understood by one of ordinary skill in the art, a gene editing system need not be a system that ‘edits’ a nucleic acid. For example, it is well recognized that a gene editing system can be used to modify target nucleic acids (e.g., DNA and/or RNA) in a variety of ways without creating a double strand break (DSB) in the target DNA. For example, in some cases a double stranded target DNA is nicked (one strand is cleaved), and in some cases (e.g., in some cases where the gene editing protein is devoid of nuclease activity, e.g., a CRISPR/Cas RNA-guided polypeptide may harbor mutations in the catalytic nuclease domains), the target nucleic acid is not cleaved at all. For example, in some cases a CRISPR/Cas protein (e.g., Cas9, CasX, CasY, Cpf1) with or without nuclease activity, is fused to a heterologous protein domain. The heterologous protein domain can provide an activity to the fusion protein such as (i) a DNA-modifying activity (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity), (ii) a transcription modulation activity (e.g., fusion to a transcriptional repressor or activator), or (iii) an activity that modifies a protein (e.g., a histone) that is associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). As such, a gene editing system can be used in applications that modify a target nucleic acid in way that do not cleave the target nucleic acid, and can also be used in applications that modulate transcription from a target DNA.

For additional information related to programmable gene editing tools (e.g., CRISPR/Cas RNA-guided proteins such as Cas9, CasX, CasY, and Cpf1, Zinc finger proteins such as Zinc finger nucleases, TALE proteins such as TALENs, CRISPR/Cas guide RNAs, PAMs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct 22;163(3):759-71; Makarova et al, Nat Rev Microbiol. 2015 Nov;13(11):722-36; Shmakov et al., Mol Cell. 2015 Nov 5;60(3):385-97; Jinek et al., Science. 2012 Aug 17;337(6096):816-21; Chylinski et al., RNA Biol. 2013 May;10(5):726-37; Ma et al., Biomed Res Int. 2013;2013:270805; Hou et al., Proc Natl Acad Sci U S A. 2013 Sep 24;110(39):15644-9; Jinek et al., Elife. 2013;2:e00471; Pattanayak et al., Nat Biotechnol. 2013 Sep;31(9):839-43; Qi et al, Cell. 2013 Feb 28;152(5):1173-83; Wang et al., Cell. 2013 May 9;153(4):910-8; Auer et. al., Genome Res. 2013 Oct 31; Chen et. al., Nucleic Acids Res. 2013 Nov 1;41(20):e19; Cheng et. al., Cell Res. 2013 Oct;23(10):1163-71; Cho et. al., Genetics. 2013 Nov;195(3):1177-80; DiCarlo et al., Nucleic Acids Res. 2013 Apr;41(7):4336-43; Dickinson et. al., Nat Methods. 2013 Oct;10(10):1028-34; Ebina et. al., Sci Rep. 2013;3:2510; Fujii et. al, Nucleic Acids Res. 2013 Nov 1;41(20):e187; Hu et. al., Cell Res. 2013 Nov;23(11):1322-5; Jiang et. al., Nucleic Acids Res. 2013 Nov 1;41(20):e188; Larson et. al., Nat Protoc. 2013 Nov;8(11):2180-96; Mali et. at., Nat Methods. 2013 Oct;10(10):957-63; Nakayama et. al., Genesis. 2013 Dec;51(12):835-43; Ran et. al., Nat Protoc. 2013 Nov;8(11):2281-308; Ran et. al., Cell. 2013 Sep 12;154(6):1380-9; Upadhyay et. al., G3 (Bethesda). 2013 Dec 9;3(12):2233-8; Walsh et. al., Proc Natl Acad Sci U S A. 2013 Sep 24;110(39):15514-5; Xie et. al., Mol Plant. 2013 Oct 9; Yang et. al., Cell. 2013 Sep 12;154(6):1370-9; Briner et al., Mol Cell. 2014 Oct 23;56(2):333-9; Burstein et al., Nature. 2016 Dec 22—Epub ahead of print; Gao et al., Nat Biotechnol. 2016 Jul 34(7):768-73; Shmakov et al., Nat Rev Microbiol. 2017 Mar;15(3):169-182; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

### Polypeptides

In some embodiments, a human HDAC4 protein or a fragment thereof may be used in place of polynucleotide sequences. The polypeptides may be joined to a wide variety of other oligopeptides or proteins for a variety of purposes. By providing for expression of the subject peptides, various post-expression modifications may be achieved. For example, by employing the appropriate coding sequences, one may provide farnesylation or prenylation. The peptides may be PEGylated, where the polyethyleneoxy group provides for enhanced lifetime in the blood stream. The peptides may also be combined with other proteins in a fusion protein, typically where the two proteins are not normally joined, such as the Fc of an IgG isotype, which may be complement binding, with a toxin, such as ricin, abrin, diphtheria toxin, or the like, or with specific binding agents that allow targeting to specific moieties on a target cell.

The human HDAC4 protein or a fragment thereof may be fused to another polypeptide to provide for added functionality, e.g. to increase the in vivo stability, or add to a transporter domain. For example, a stable plasma protein can extend the in vivo plasma half-life of the human HDAC4 protein or a fragment thereof when present as a fusion, in particular wherein such a stable plasma protein is an immunoglobulin constant domain.

The human HDAC4 protein or a fragment thereof for use in the subject methods may be produced from eukaryotic or prokaryotic cells or may be synthesized in vitro. Where the protein is produced by prokaryotic cells, it may be further processed by unfolding, e.g. heat denaturation, DTT reduction, etc. and may be further refolded, using methods known in the art. By using synthesizers, naturally occurring amino acids may be substituted with unnatural amino acids. The particular sequence and the manner of preparation will be determined by convenience, economics, purity required, and the like.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the formulations which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

### Pharmaceutical Compositions

For administration to a subject such as a human or other mammal (e.g., companion, zoological or livestock animal), human HDAC4 protein or a fragment thereof is desirably formulated into a pharmaceutical composition containing the active agent in admixture with one or more pharmaceutically acceptable diluents, excipients or carriers. Examples of such suitable excipients for can be found in U.S. Publication 2009/0298785 (incorporated by reference herein in its entirety), the Handbook of Pharmaceutical Excipients, 2nd Edition (1994), Wade and Weller, eds. Acceptable carriers or diluents for therapeutic use are well-known in the pharmaceutical art, and are described, for example, in Remington: The Science and Practice of Pharmacy, 20th Edition (2000) Alfonso R. Gennaro, ed., Lippincott Williams & Wilkins: Philadelphia, Pa.

The choice of pharmaceutical carrier, excipient or diluent can be selected with regard to the intended route of administration and standard pharmaceutical practice. The pharmaceutical composition can contain as, or in addition to, the carrier, excipient or diluent any suitable binder(s), lubricant(s), suspending agent(s), coating agent(s), solubilizing agent(s).

A person of ordinary skill in the art can easily determine an appropriate dosage to administer to a subject without undue experimentation. Typically, a physician will determine the actual dosage that will be most suitable for an individual subject based upon a variety of factors including the activity of the specific compound employed, the metabolic stability and length of action of the compound, the age, body weight, general health, diet, mode and time of administration, rate of excretion, drug combination, the severity of the particular condition, and the individual undergoing therapy. To determine a suitable dose, the physician or veterinarian could start doses levels lower than that required in order to achieve the desired therapeutic effect and gradually increase the dosage until the desired effect is achieved. This is considered to be within the skill of the artisan and one can review the existing literature on a specific agent to determine optimal dosing.

In some embodiments, the composition is administered in the form of a liquid (e.g., drop or spray) or gel suspension. Alternatively, the composition is applied to the eye via liposomes or Infused into the tear film via a pump-catheter system. Further embodiments embrace a continuous or selective-release device, for example, membranes such as, but not limited to, those employed in the OCUSERT System (Alza Corp., Palo Alto, Calif.) in an alternative embodiment, the human HDAC4 protein or a fragment thereof composition is contained within, carried by, or attached to a contact lens, which is placed on the eye. Still other embodiments embrace the use of the composition within a swab or sponge, which is applied to the ocular surface.

In some cases, the composition further comprises a pharmaceutically acceptable carrier, e.g., a pharmaceutically acceptable salt. Suitable ocular formulation excipients include FDA approved ophthalmic excipients, e.g., emulsions, solutions, solution drops, suspensions, and suspension drops. Other suitable classifications include gels, ointments, and inserts/implants.

Exemplary excipients for use in optimizing ocular formulations include alcohol, castor oil, glycerin, polyoxyl 35 castor oil, Tyloxapol, polyethylene glycol 8000 (PEG-8000), ethanol, glycerin, cremaphor, propylene glycol (pG), polypropylene glycol (ppG), and polysorbate 80. In some cases, citrate buffer and sodium hydroxide are included to adjust pH. Preferably, the formulation for ocular delivery of nutlin-3a comprises 5% cremaphor, 10% pG, 15% pPG, and 70% phosphate buffered saline (PBS).

In certain embodiments, multiple therapeutically effective doses are administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, once every two weeks, once every three weeks, once a month, and so forth. For example, in some embodiments, an antibody is administered once every two to four weeks for an extended period of time, such as for 1, 2, 3, 4, 5, 6, 7, 8, 10, 15, 24 months, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted herein below.

The therapeutic dose may be at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 μg/kg body weight. It will be understood by one of skill in the art that such guidelines will be adjusted for the molecular weight of the active agent. The dosage may also be varied for localized administration, e.g. intravitreal, inhalation, etc., or for systemic administration, e.g. i.m., i.p., i.v., and the like.

In certain embodiments, the presently disclosed subject matter also includes combination therapies. Depending on the particular disease, disorder, or condition to be treated or prevented, additional therapeutic agents, which are normally administered to treat or prevent that condition, may be administered in combination with the compounds of this disclosure. These additional agents may be administered separately, as part of a multiple dosage regimen. Alternatively, these agents may be part of a single dosage form, mixed together with the human HDAC4 protein or a fragment thereof, or polynucleotides encoding human HDAC4 protein or a fragment thereof.

By “in combination with” is meant the administration of an agent, or other compounds disclosed herein, with one or more therapeutic agents either simultaneously, sequentially, or a combination thereof. Therefore, a cell or a subject administered a combination of an agent, can receive one or more therapeutic agents at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the agent and one or more therapeutic agents are administered simultaneously, they can be administered to the cell or administered to the subject as separate pharmaceutical compositions or they can contact the cell as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered compound is not diminished by the sequential, simultaneous or separate administration of the subsequent compound(s).

Also provided are a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In some embodiments, the kits comprise one or more containers, including, but not limited to a vial, tube, ampule, bottle and the like, for containing the compound. The one or more containers also can be carried within a suitable carrier, such as a box, carton, tube or the like. Such containers can be made of plastic, glass, laminated paper, metal foil, or other materials suitable for holding medicaments.

In some embodiments, the container can hold a composition that is by itself or when combined with another composition effective for treating or preventing the condition and may have a sterile access port (for example the container may be an intravenous solution bag or a vial having a stopper pierceable by a hypodermic injection needle). Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The presently disclosed kits or pharmaceutical systems also can include associated instructions for using the compounds for treating or preventing a neurodegenerative disease, disorder, or condition, e.g. optic neuritis, including glaucoma. In some embodiments, the instructions include one or more of the following: a description of the active compound; a dosage schedule and administration; precautions; warnings; indications; counter-indications; overdosage information; adverse reactions; animal pharmacology; clinical studies; and references. The instructions can be printed directly on a container (when present), as a label applied to the container, as a separate sheet, pamphlet, card, or folder supplied in or with the container.

### EXPERIMENTAL

Glaucoma, characterized by neurodegeneration of retinal ganglion cells (RGCs), is the leading cause of irreversible blindness in the world. Currently, the main therapeutic approach for glaucoma is lowering intraocular pressure (IOP). However, this strategy is not effective in all patients with glaucoma.

RGC death and survival is a complex process that is controlled in part by changes in gene expression, including that regulated by histone deacetylases (HDACs). There are 18 different human HDACs that are typically divided into 4 major classes. The focus of this disclosure is the type 4 class IIa HDAC (HDAC4) that our data show is neuroprotective for RGCs. In general, HDACs catalyze the removal of acetyl groups from substrate proteins, such that their canonical function is to repress gene transcription by histone deacetylation. We have found that depletion of HDAC4 exacerbated RGC death after optic nerve injury, while increased nuclear expression of HDAC4 enhanced RGC survival. We disclose how HDAC4 promotes RGC survival and axon regeneration, providing a new approach to the treatment of neurodegenerative diseases such as glaucoma.

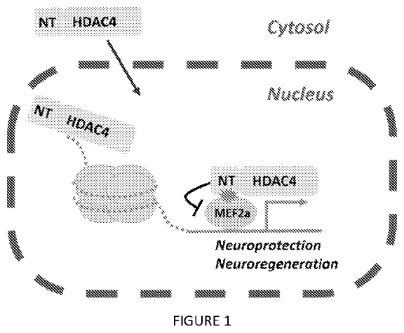

The canonical function of class IIa HDACs is to alter chromatin structure and repress gene expression via binding to site-specific transcription factors (TFs), among which myocyte enhancer factor 2 (MEF2) family members are the best characterized. MEF2 family members have been shown to play a pivotal role in neurons. Notably, recent studies showed that MEF2A gene deletion mice enhanced RGC survival after optic nerve crush injury. Class IIa HDACs tend to have relatively low deacetylase activity and repress gene expression in conjunction with other additional mechanisms, including, for example, the recruitment (scaffolding) of other gene-regulating enzymes. Interestingly, MEF2a transcriptional activity is activated by desumoylation at Lys-403 following adjacent Ser-408 phosphorylation, while HDAC4 binding is associated with MEF2A sumoylation. In myocytes an N-terminal HDAC4 fragment (HDAC4-NT) is proteolytically released through a PKA-dependent mechanism. HDAC4-NT specifically inhibits MEF2 transcriptional activity and does not bind other MADS domain transcription factors like serum response factor. Based upon the regulation of MEF2a and data showing the beneficial effects of a nuclear localized HDAC4 mutant and the HDAC4-NT fragment, we find that nuclear HDAC4 protects retinal ganglion cells via repression of MEF2a-dependent gene expression (FIG. 1).

### Results

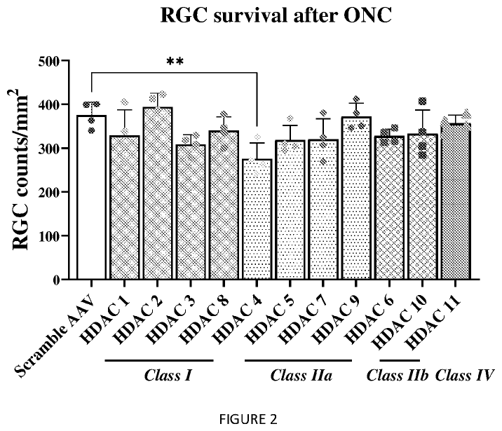

HDAC4 depletion significantly decreases RGC loss after optic nerve injury. To screen for the relevance of HDACs to RGC death and survival in an in vivo disease model, we have expressed in RGCs using adeno-associated virus (AAV) small hairpin RNAs (shRNA) for each HDAC isoenzyme. RGC survival after optic nerve crush (ONC) injury was that induces severe axon damage and RGC loss (FIG. 2). AAV2.HDAC-shRNA vectors were intravitreally injected into C57BL/6J (not shown) and S129 mice, obtaining similar results. Two weeks after optic nerve crush injury, by when 80-90% of RGCs are typically lost, retina flat mounts were stained with the RGC marker RBPMS to quantify RGC survival. Inhibited expression of no individual HDAC significantly improved RGC survival after crush injury. Instead, the inhibited expression of only HDAC4 significantly decreased RGC survival.

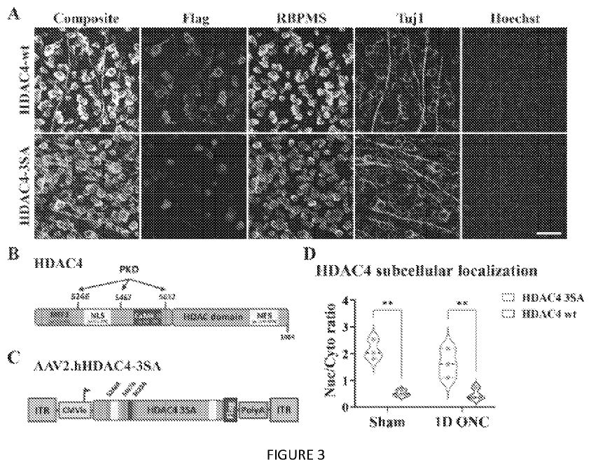

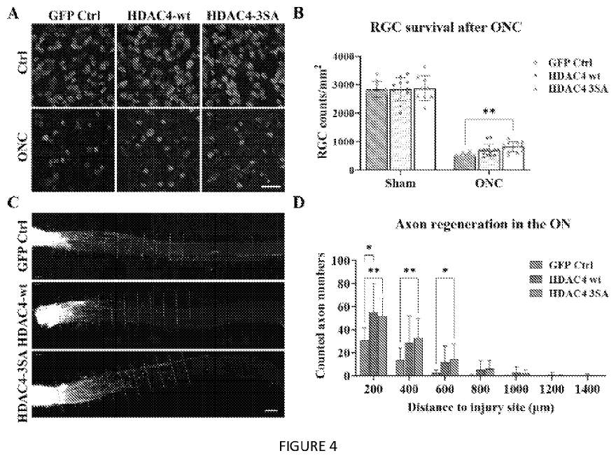

Nuclear-localized HDAC4 protects RGCs after injury. Class IIa HDACs (4,5,7,9), share a common structure with a C-terminal catalytic domain and an N-terminal regulatory domain that interacts with transcription factors, coactivators, and corepressors (FIG. 3B). The subcellular localization of class IIs HDACs is regulated by phosphorylation, including by PKD that promotes binding by 14-3-3 chaperone proteins; 14-3-3 escorts phosphorylated HDAC from the nucleus into the cytoplasm, activating target genes. To test whether nuclear HDAC4, that promotes gene repression, might preferentially enhance RGC survival, we expressed a nuclear localized, phosphoablative HDAC4 mutant for the PKD phosphorylation sites S246, S467, and S632 (3SA, FIG. 3C). Flag-tagged wildtype and mutant HDAC4 expressed using AAV2 were detected in retina by immunohistochemistry 1 day after ONC or sham-operation. Flag-HDAC4-3SA was localized preferentially to the nucleus (nuclear/cytosol ratio >1), whereas wildtype Flag-HDAC4 was mainly cytosolic (nuclear/cytosol ratio <1, FIG. 3A, D). Notably, HDAC4-3SA significantly protected RGC 2 weeks after ONC (FIG. 4A-B). Overexpression of wild type HDAC4 also tended to increase RGC survival after injury, although not reaching statistical difference. Cholera toxin staining for axons in the optic nerve revealed that the nuclear localized HDAC4 mutant also promoted more axon regeneration than GFP control. (FIG. 4C-D).

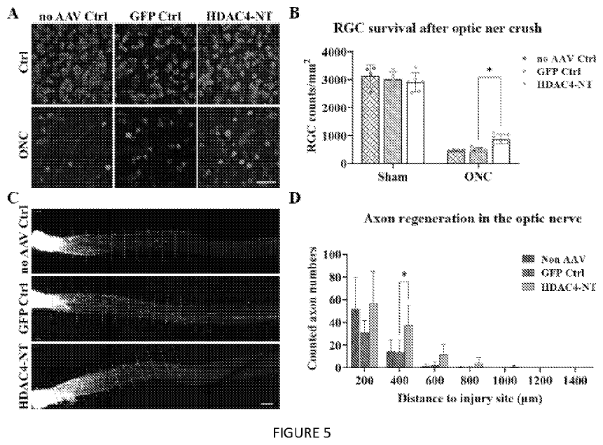

Expression of the HDAC4 N-terminal domain is neuroprotective after injury. Studies in myocytes showed that HDAC4 is proteolytically processed by a PKA dependent mechanism to yield an active N-terminal fragment (HDAC4-NT). HDAC4-NT selectively inhibits the activity of the transcription factor myocyte enhancer factor 2 (MEF2). In contrast to full-length HDAC4, which is regulated in cytoplasmic-nuclear localization, HDAC4-NT is a constitutive repressor of MEF2-dependent gene expression. To test whether the neuroprotective effects of HDAC4 might be conferred by repression of MEF2-regulated genes, we expressed HDAC4-NT by AAV2 intravitreal injection of C57BL/6J mice. In comparison to non-injected and AAV2-GFP injected control retinas, there was greater RGC survival following HDAC4-NT expression. In addition, we found that HDAC4-NT expression promoted axon regeneration 2 weeks after ONC injury. (FIG. 5).

### References

Causes of blindness and vision impairment in 2020 and trends over 30 years, and prevalence of avoidable blindness in relation to VISION 2020: the Right to Sight: an analysis for the Global Burden of Disease Study. Lancet Glob Health, 2021. 9(2): p. e144-e160.

Sando, R., 3rd, et al., HDAC4 governs a transcriptional program essential for synaptic plasticity and memory. Cell, 2012. 151(4): p. 821-834.

Bertos, N.R., A.H. Wang, and X.J. Yang, Class II histone deacetylases: structure, function, and regulation. Biochem Cell Biol, 2001. 79(3): p. 243-52.

Di Giorgio, E. and C. Brancolini, Regulation of class IIa HDAC activities: it is not only matter of subcellular localization. Epigenomics, 2016. 8(2): p. 251-69.

Zhu, B., et al., The transcription factor MEF2A plays a key role in the differentiation/maturation of rat neural stem cells into neurons. Biochem Biophys Res Commun, 2018. 500(3): p. 645-649.

Pulimood, N.S., et al., The Role of CREB, SRF, and MEF2 in Activity-Dependent Neuronal Plasticity in the Visual Cortex. J Neurosci, 2017. 37(28): p. 6628-6637.

Dietrich, J.B., The MEF2 family and the brain: from molecules to memory. Cell Tissue Res, 2013. 352(2): p. 179-90.

Xia, X., et al., MEF2 transcription factors differentially contribute to retinal ganglion cell loss after optic nerve injury. PLoS One, 2020. 15(12): p. e0242884.

Welsbie, D.S., et al., Enhanced Functional Genomic Screening Identifies Novel Mediators of Dual Leucine Zipper Kinase-Dependent Injury Signaling in Neurons. Neuron, 2017. 94(6): p. 1142-1154 e6.

Shalizi, A., et al., A calcium-regulated MEF2 sumoylation switch controls postsynaptic differentiation. Science (New York, N.Y.), 2006. 311(5763): p. 1012-7.

Backs, J., et al., Selective repression of MEF2 activity by PKA-dependent proteolysis of HDAC4. J Cell Biol, 2011. 195(3): p. 403-15.

Veloso, A., et al., Dephosphorylation of HDAC4 by PP2A-Bδ unravels a new role for the HDAC4/MEF2 axis in myoblast fusion. Cell Death Dis, 2019. 10(7): p. 512.

Miska, E.A., et al., HDAC4 deacetylase associates with and represses the MEF2 transcription factor. Embo j, 1999. 18(18): p. 5099-107.

Youn, H.D., C.M. Grozinger, and J.O. Liu, Calcium regulates transcriptional repression of myocyte enhancer factor 2 by histone deacetylase 4. J Biol Chem, 2000. 275(29): p. 22563-7.

Zhao, X., et al., Regulation of MEF2 by histone deacetylase 4-and SIRT1 deacetylase-mediated lysine modifications. Mol Cell Biol, 2005. 25(19): p. 8456-64.

Martin, M., R. Kettmann, and F. Dequiedt, Class IIa histone deacetylases: regulating the regulators. Oncogene, 2007. 26(37): p. 5450-67.

Di Giorgio, E., et al., MEF2 is a converging hub for histone deacetylase 4 and phosphatidylinositol 3-kinase/Akt-induced transformation. Mol Cell Biol, 2013. 33(22): p. 4473-91.

Grozinger, C.M. and S.L. Schreiber, Regulation of histone deacetylase 4 and 5 and transcriptional activity by 14-3-3-dependent cellular localization. Proc Natl Acad Sci U S A, 2000. 97(14): p. 7835-40.

Park, S.-Y. and J.-S. Kim, A short guide to histone deacetylases including recent progress on class II enzymes. Experimental & Molecular Medicine, 2020. 52(2): p. 204-212.

Lehmann, L.H., et al., A proteolytic fragment of histone deacetylase 4 protects the heart from failure by regulating the hexosamine biosynthetic pathway. Nat Med, 2018. 24(1): p. 62-72.

1. A method of treating glaucoma or other optic neuropathies, the method comprising:
delivering to a retinal ganglion cell in the eye of a subject, a composition comprising a polynucleotide sequence that encodes human HDAC4 protein or a fragment thereof, operably linked to a promoter active in the cell;
thereby preventing or reducing degeneration or vision loss in glaucoma or other optic neuropathies.

2. The method of claim 1, wherein the HDAC4 is a nuclear localized, phosphoablative HDAC4 mutant.

3. The method of claim 1, wherein the HDAC4 mutant comprises amino acid substitutions at one or more residues selected from S246, S467 and S632, where the amino acid substitutions are to an amino acid other than serine, and numbering is made relative to SEQ ID NO: 1.

4. The method of claim 1, wherein the HDAC4 mutant comprises each of the amino acid substitutions S246A, S467A, and S632A.

5. The method of claim 1, wherein the HDAC4 fragment is an N-terminal fragment.

6. The method of claim 5, wherein the N-terminal fragment is truncated at about residue 201, relative to SEQ ID No. 1.

7. The method of claim 1, wherein the composition is administered intravitreally, subretinally, subconjunctivally, or intravenously.

8. The method of claim 1, wherein the subject is a human.

9. The method of claim 1, wherein the polynucleotide sequence is in operable linkage with a human cytomegalovirus immediate early enhancer/promoter.

10. The method of claim 1, wherein the polynucleotide comprises a guide RNA specific for human HDAC4, wherein CRISPR is used to mutate RGC HDAC4 to a nuclear localized, phosphoablative HDAC4 mutant or to an N-terminal fragment of HDAC4.

11. The method of claim 1, wherein the polynucleotide is used for genomic modification to increase expression of HDAC4.

12. The method of claim 1, wherein the polynucleotide sequence further comprises AAV2 inverted terminal repeats.

13. The method of claim 12, wherein the polynucleotide is packaged in an AAV particle.

14. The method of claim 1, wherein the composition further comprises a pharmaceutically acceptable diluent, excipient, vehicle, or carrier.

15. The method of claim 1, wherein the composition further comprises a second therapeutic agent for the treatment of glaucoma and other optic neuropathies.

16. A composition comprising a polynucleotide sequence that encodes human HDAC4 protein or a fragment thereof, operably linked to a promoter active in a neuronal cell for use in the methods of any of claims 1.


SEQUENCE LISTING (1 sequence)

SEQ ID NO: 1 - protein, 1084 aa, Homo sapiens
MSSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDAERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL